(19) United States
(12) Patent Application Publication
(10) Pub. No.: US 2024/0348873 A1
(43) Pub. Date: Oct. 17, 2024

(54) METHOD OF AND DEVICE FOR MONITORING ONE OR MORE PHYSIOLOGICAL PARAMETERS

(71) Applicants: Sony Group Corporation, Tokyo (JP); Sony Europe B.V., Surrey (GB)

(72) Inventors: Andres Jose ROMAN, Barcelona (ES); Renaud DI FRANCESCO, London (GB)

(21) Appl. No.: 18/579,808

(22) PCT Filed: Jul. 6, 2022
(86) PCT No.: PCT/GB2022/051728

§ 371 Date: Jan. 16, 2024

(30) Foreign Application Priority Data
Jul. 30, 2021 (GB) 2110991.3

(51) Int. Cl. H04N 21/442; H04N 21/466

(52) U.S. Cl. CPC H04N 21/44218; H04N 21/4668

(57) ABSTRACT

A method of monitoring one or more physiological parameters of a consumer of audio and/or visual content, the method comprising: determining one or more expected physiological parameter values of the consumer when consuming the content based on a classification of the content; comparing the one or more expected physiological parameter values with one or more corresponding actual physiological parameter values of the consumer when consuming the content; performing a process in response to one or more of the one or more actual physiological parameter values differing from its corresponding expected physiological parameter value.

### BACKGROUND

### Field of the Disclosure

The present disclosure relates to a method of and device for monitoring one or more physiological parameters.

### Description of the Related Art

The “background” description provided is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present disclosure.

Modern technology allows various physiological parameters of a person such as heart rate, blood pressure and the like to be determined quickly, easily and with high levels of accuracy. Known techniques such as remote photoplethysmography (PPG) even allow changes in blood flow to be detected in moving images of a person based on subtle changes to the skin colour or changes in the phase of light waves reflected from the person's skin, for example. This allows parameters such as heart rate and blood pressure to be determined without even having to make contact with the person's skin.

Much of this technology was developed and is used in conventional clinical settings. However, as the technology underlying these techniques (for example high quality image sensors and the like) becomes more commonplace, there is a desire to make this technology more widely available as an indicator of patient health in everyday life.

### SUMMARY

The present disclosure is defined by the claims.

### BRIEF DESCRIPTION OF THE DRAWINGS

Non-limiting embodiments and advantages of the present disclosure are explained with reference to the following detailed description taken in conjunction with the accompanying drawings, wherein:

FIG. 1 shows an example device according to embodiment(s);

FIG. 2 shows some internal components of the example device of FIG. 1;

FIG. 3 shows an example implementation according to embodiment(s);

FIG. 4 shows an example initial correlation data set;

FIGS. 5A to 5D show an example user set up process;

FIGS. 6A to 6B show a first example viewer alert;

FIGS. 7A to 7B show a second example viewer alert;

FIGS. 8A to 8B exemplify generation of an updated correlation data set;

FIG. 9 shows an example method according to embodiment(s); and

FIG. 10 shows an example system according to embodiment(s).

Like reference numerals designate identical or corresponding parts throughout the drawings.

### DETAILED DESCRIPTION OF THE EMBODIMENTS

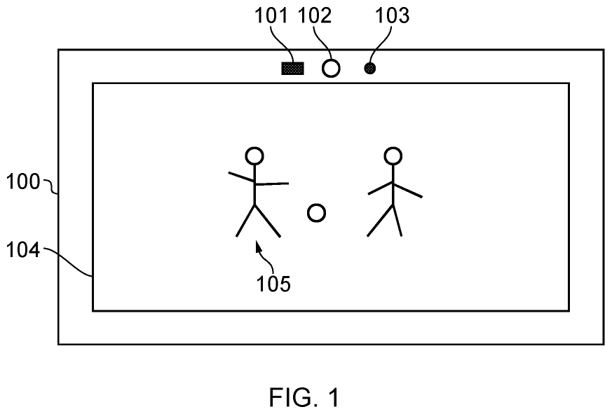

FIG. 1 shows a television (TV) 100 as an example of a device/apparatus according to the present disclosure. The TV 100 comprises a screen (for example a liquid crystal display (LCD) or organic light emitting diode (OLED) display) for displaying electronic images 105 to a viewer. The TV may also comprise a loudspeaker (not shown) for outputting sound to the viewer or viewers.

The TV 100 comprises several sensors. In this example, the sensors include a camera 102, a time-of-flight (TOF) sensor 101 and a microphone 103. In some embodiments the apparatus 100 may be a system of components including a screen, camera, TOF sensor, microphone and content receiver such as a set top box, connected via an appropriate protocol such as a wireless protocol, HDMI and/or USB interconnection or proprietary interconnection.

The camera 102 comprising one or more lenses (not shown) which focus incident light onto an image sensor (for example a complementary metal-oxide-semiconductor (CMOS)) to allow electronic images of the field of view of the camera 102 to be captured and stored. The position of the camera means it can capture a live moving image of the viewer as they watch the TV screen 104.

The TOF sensor 101 emits pulses of light (for example infrared (IR) laser or LED light) over the scene and detects each pulse of light again when it is reflected back from an object in the scene. By measuring the time between emitting each pulse of light and detecting it again, the TOF sensor is able to determine the distance the light travels. By emitting and detecting many (for example several thousands of) pulses of light over different parts of the scene every second, a detailed depth map of objects in the scene can be generated. The position of the TOF sensor means it can capture a live moving depth map of the viewer as they watch the TV screen 104.

The microphone 103 captures ambient sounds in the vicinity of the TV 100. It may employ a filter to filter out sounds emitted by the TV's loudspeaker to alleviate unwanted feedback (for example by superposing a 180° out-of-phase version of the sound signal output by the loudspeaker onto the detected ambient sound signal). It may also be a directional microphone so that only sounds emitted from a certain direction with respect to the position and orientation of the microphone are detected. This can be achieved, for example, by using a grid of transducers such that destructive interference is exhibited on signals generated from sounds in unwanted directions. The position of the microphone (and its directional configuration, if it is a directional microphone) means it can capture sounds made by the viewer (for example speech or breathing sounds).

Sensors such as the camera 102, TOF sensor 101 and microphone 103 are known in the art and are available from various manufacturers such as Sony®. They are therefore not discussed in detail here.

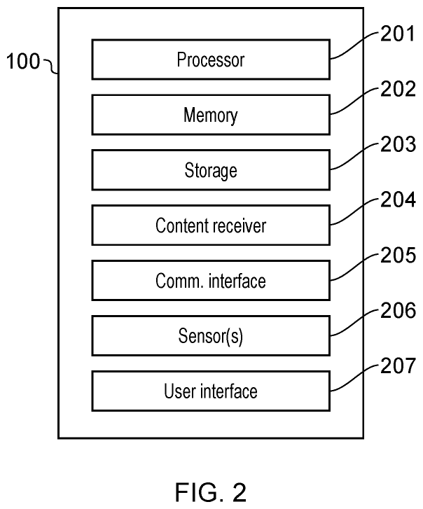

FIG. 2 shows some internal components of TV 100. The TV comprises a processor 201 for executing electronic instructions, a memory 202 for storing the electronic instructions to be executed and electronic input and output data associated with the electronic instructions, a storage medium 203 (for example a solid state drive) for long term storage of electronic data, a content receiver 204 for receiving audio-visual (AV) content to be output to a viewer by the TV, a communication interface 205 for sending electronic data to and/or receiving electronic data from one or more other devices (for example over a network such as the internet), one or more sensors 206 (comprising for example the camera 102, TOF sensor 101 and microphone 103 of FIG. 1) and a user interface 207 (comprising for example the screen 104 of FIG. 1 and a TV remote control (not shown)) for outputting information to and/or receiving commands from the viewer. Each of the processor 201, memory 202, storage medium 203, content receiver 204, communication interface 205, one or more sensors 206 and user interface 207 are implemented using appropriate circuitry, for example. The processor 201 controls the operation of each of the memory 202, storage medium 203, content receiver 204, communication interface 205, one or more sensors 206 and user interface 207.

In an example, the sensors 206 may comprise one or more further types of sensor, for example a wearable sensor (such as an electrocardiograph (ECG) sensor, oxygen saturation probe, wearable heart rate monitor, wearable blood pressure monitor or wearable monitor for measuring respiratory information (such as breathing rate)) or a non-wearable sensor (such as a thermal imaging camera for detecting body temperature). Wearable sensors may transmit sensor data for processing by the processor 201 via the communication interface 205 (using, for example, a direct connection to the communication interface 205 or a connection to the communication interface 205 via one or more servers on a network such as the internet.

In an example, the content receiver 204 comprises a radio frequency (RF) receiver such as a Digital Video Broadcasting (DVB) receiver for receiving broadcast content via radio signals. In another example, the content receiver 204 comprises a network interface configured to receive digital content over a network such as the internet. For example, the content may be over-the-top (OTT) or IPTV (Internet Protocol Television) content. It may be scheduled or on-demand. In the latter case, the function of the content receiver 204 may be implemented by the communication interface 205 (meaning a standalone content receiver 204 is not required).

Although the device 100 is a TV in this case, the device 100 may instead be a separate device which is connectable to a TV, either externally by the viewer (via for example a High-Definition Media Interface (HDMI) or Universal Serial Bus (USB) connection) or internally by a TV manufacturer. In this case, the content may be received by the content receiver 204 after it has been received by the TV. In the case in which the device 100 is an external device, the content receiver 204 comprises the connection to the TV (e.g. HDMI and/or USB connection) which enables content received for display on the TV to be received by the device 100. Furthermore, the user interface 207 may receive commands from the viewer via, for example, voice commands detected by the microphone 103 and/or a separate remote control (not shown) and the user interface 207 may output information to the viewer by transmitting electronic signals to the TV (which the TV then outputs via its screen and/or loudspeaker). In the case of an external device, the external device is configured to be positioned so that its sensor(s) (for example camera 102, TOF sensor 101 and microphone 103) face the viewer when they are watching the TV, for example. In the case of an internal device, the sensor(s) are positioned within a housing of the TV so they face the viewer when they are watching the TV, for example.

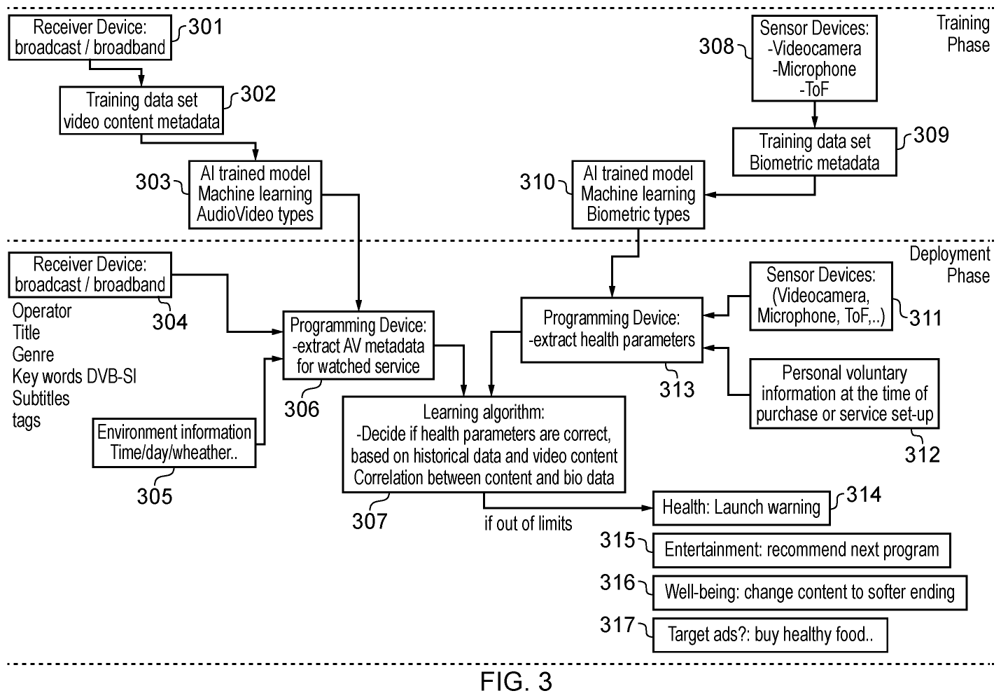

FIG. 3 shows an example implementation of the present disclosure in which the sensor(s) 206 are used to detect the values of one or more physiological (biometric) parameters of the viewer. The detected values are then compared to expected values of those physiological parameter(s) so as to be able to learn information about the viewer's health. For example, based on a remote PPG technique applied to a moving image captured by the camera 102 and/or a moving depth map captured by the TOF sensor 101, the viewer's heart rate and blood pressure may be determined and compared to respective expected value ranges. If the measured heart rate and/or blood pressure lie outside the respective expected value ranges, an alert is issued to the user and/or a third party (for example healthcare professional) indicating the discrepancy.

More generally, the detected physiological parameter value(s) allow the device 100 to learn information about the viewer's health. When the detected values are compared to the corresponding expected values for a given viewer, this allows active monitoring of the viewer's health and, in an example, may be used to help determine the current health status of the viewer and diagnose any health condition they have. In particular, detected deviations from the expected values may be used to help decide on the need for further and/or deeper investigations in a more comprehensive medical environment.

The present disclosure, however, recognises that the expected value of a given physiological parameter will differ depending on the type of content the user is watching. For example, if it is noted the viewer has a very high heart rate but they are watching an exercise video, the chance is the user is not experiencing a health issue because it is expected their heart rate would show a significant increase in this scenario (because they are likely to be exercising). An alert is therefore not issued in this case. On the other hand, if the same very high heart rate is detected when the user is watching a documentary, there is a greater chance the user is experiencing a health issue because it is not expected their heart rate would be so high in this scenario (because they are likely sitting down). The present disclosure thus helps distinguish between scenarios such as this so as to undertake more appropriate health interventions on behalf of the viewer.

FIG. 3 shows a training phase and a deployment phase. The training phase involves training two machine learning models, a content type (CT) model and a physiological parameter (PP) model.

Steps 301, 302 and 303 relate to training the CT model.

At step 301, different instances of video content are obtained (in this example, via radio broadcast or internet broadband). The video content is of various different types with different types of content having different characteristics which allow that type of content to be identified. For example, the different types of video content may include different genres of content (for example drama, comedy, sport, news, documentary) from different types of content providers (for example public service or private service broadcasting). The characteristics which allow the different types of content to be recognised could be broadcast attributes provided with the content in the form of metadata (indicating for example the operator, channel or service, title, genre, keywords (as indicated in the DVB Service Information (SI), for example), subtitles, event table descriptor, electronic program guide (EPG, subject to agreement with the EPG provider), an indicator of whether the content is a public service or private service broadcast and/or any additional metadata tags) or could be characteristics derivable from image and/or sound data of the content itself (for example average volume, type of sound (for example speech or music), colour palette, amount of motion, etc.). It is envisaged any type of characteristic which allows classification of a given instance of content into one of a plurality of content classifications could be used. Corresponding attributes to those available for DVB are available for IPTV or OTT services. Attributes may be provided separately to the content in an internet delivered EPG arranged in a structured way. In some embodiments public service content may be considered more trusted or at least more predictable. Some services or channels (and these may include public service content) may deliver content that might be more risqué than the norm and be relatively more likely to expose certain physiological characteristics, or indeed false positive alerts.

Content may also be supplied with metadata indicating the content is suitable for use in monitoring viewer physiological parameter value(s) for medical purposes. For example, a public service broadcast drama with well-known characters and a consistent storyline may be particularly suitable for determining health problems in an elderly viewer (for example) since that viewer is expected to be relaxed whilst watching such content. If there are indicators of stress (for example a spike in heart rate and/or blood pressure), this is more likely to be a result of a problem rather than simply a reaction to the content. Content such as this may therefore be tagged with metadata (for example a predetermined flag) indicating a deviation from the expected physiological parameter value(s) for that content should be taken particularly seriously. For example, if the expected physiological parameter value(s) are exceeded for this content, a responsive process may be to alert a third party (for example a medical professional) rather than only alerting the viewer themselves.

More generally, with examples of the present disclosure, the content may set a context for measurement of viewer physiological parameter(s). For example, to define the context, the content can be categorised with metadata in qualitative and/or quantitative terms (for example, a rating of one (mild), two (moderate) and three (high) for features of the content such as the level of mystery, violence, emotions, etc.). The context information may then be made available to, for example, a medical professional associated with the viewer (for example, the viewer's GP) together with the associated measured physiological parameter value(s) of the viewer when viewing the content.

This allows the medical professional to review the physiological parameter data in the context of the type of content the viewer is watching and the expected physiological parameter value(s) for that content (the expected values being based on, for example, physiological data pooled from a sample of viewers and/or historical physiological data of the viewer themselves). Thus, for example, it may be normal for a viewer's heart rate to increase significantly while viewing content with a high mystery and/or violence rating (and thus the medical professional may decide not to take any action to address such an increase in this scenario) but not normal for a viewer's heart rate to increase significantly while viewing content with a low mystery and violence rating (and thus the medical professional may decide to take an appropriate action to address such an increase in this scenario, for example, adjusting the viewer's medication).

At step 302, the received instances of video content (each instance of video content being, for example, a video clip of a given program of a given time duration (for example 10 seconds)) are collected to form a training set. The training set comprises a sufficient number of instances of content of each classification to enable a machine learning classification model to be adequately trained (so it is able to classify new instances of content it has not seen before into the correct category with an acceptable accuracy, for example 80%). In an example, 5,000 instances of content in each category are used. 4,000 of these instances are randomly selected for inclusion in the training set and 1,000 are used in a test set to test the model and optimise the model hyper-parameters. Thus, for example, if there are 10 different content classifications (for example drama (public service broadcast), drama (private service broadcast), comedy, sport, etc.), 50,000 instances of content are used to train and test the model (with a total of 40,000 instances in the training set and 10,000 instances in the test set). The classification of each instance of content in the training set is known to the model. The classification of each instance of content in the test set is not known to the model. This enables the performance of the model to be tested using the test set once it has been trained using the training set. It is noted the number of instances of training data and test data and the acceptable accuracy given here represent an example only and that, in reality, the skilled person will choose these values according the nature of the model to be trained, the statistical nature of the data set, etc. It is also noted that, as the system is rolled out, the CT model can continue to be trained and refined to improve its accuracy. For example, a random sample of real life viewers may be asked at particular intervals to manually classify a particular instance of content they are watching and this can then be used as additional training data for the CT model. In this way, the accuracy of the CT model be improved as it is used in real life.

At step 303, the model is trained and tested using the training set and the test set. Any suitable machine learning classification model may be used, with the chosen model depending on the characteristics of the content used for classification. As is the norm for machine learning, the skilled person may choose a plurality of different models (and, where applicable, configurations of each model), train each one using the training set and test which of these gives the most accurate classification results using the test set. Example machine learning models which could be used include logistic regression, K-Nearest Neighbours (K-NN), Support Vector Machine (SVM), Kernel SVM, Naïve Bayes, Decision Tree Classification, Random Forest Classification, Deep Learning (including Artificial Neural Networks (ANNs) and, in for example the case of image classification, Convolutional Neural Networks (CNNs)) and Reinforcement Learning.

Once trained, the CT model is stored either locally in the memory 202 and/or storage medium 203 of the device 100 or is stored on a remote device (for example a server) accessible to the device 100 via the communication interface 205 (for example using a suitable Application Programming Interface (API)). Once trained, the CT model is able to take, as input, an instance of content it has not seen before (for example a 10 second clip of content received by content receiver 204) and, as output, classify it into one of the plurality of predetermined content classifications used in the training.

Steps 308, 309 and 310 relate to training the PP model.

At step 308, different instances of user data are obtained. The user data is, for example, moving image, depth map and/or audio data of users captured using sensor(s) such as the camera 102, TOF sensor 101 and microphone 103. The user data is of different types of users (for example of different ages, genders, fitness levels) in different states of physical and/or psychological exertion (for example exercising, standing still, sitting, lying down, concentrating, relaxed) and each instance of user data (for example a combined capture of video, depth map and/or audio data over a given time period, for example 10 seconds, for a given user) is associated with corresponding physiological data measured using a suitable known technique (for example an electrocardiographic monitor for measuring heart rate, a sphygmomanometer for measuring blood pressure and data manually collected by a medical expert such as a neurologist to indicate a type of movement the user is undergoing).

At step 309, the received instances of user data (each instance of user data being a set of data collected from a given user over a given time period) are collected to form a training set. The training set comprises a sufficient number of instances of user data of different types of users in different states of physical and/or psychological exertion to enable a machine learning classification and/or regression model to be adequately trained (so it is able to determine the physiological characteristics of new instances of user data with an acceptable accuracy, for example 80% correct classification or regression to within 20% of the correct value). In an example, 5,000 instances of user data in each of a number of user categories (for example person in age group 18-35 exercising, person in age group 18-35 standing still, person in age group 18-35 sitting, person in age group 18-35 lying down, person in age group 36-65 exercising, etc.) are used. 4,000 of these instances are randomly selected for inclusion in the training set and 1,000 are used in a test set to test the model and optimise the model hyper-parameters. Thus, for example, if there are 10 user categories, 50,000 instances of user data are used to train and test the model (with a total of 40,000 instances in the training set and 10,000 instances in the test set). The measured physiological parameter values (for example heart rate, blood pressure, movement type) of each instance of user data in the training set is known to the model. The measured physiological parameter values of each instance of user data in the test set is not known to the model. This enables the performance of the model to be tested using the test set once it has been trained using the training set. It is noted the number of instances of training data and test data and the acceptable accuracy given here represent an example only and that, in reality, the skilled person will choose these values according the nature of the model to be trained, the statistical nature of the data set, etc. It is also noted that, as the system is rolled out, the PP model can continue to be trained and refined to improve its accuracy. For example, a random sample of real life viewers may be asked at particular intervals to manually provide physiological parameter measurement(s) whilst they are watching content of a particular content classification (e.g. using an independent physiological parameter measurement method to the sensor(s) 206) and this can then be used as additional training data for the PP model. In this way, the accuracy of the PP model be improved as it is used in real life.

At step 310, the model is trained and tested using the training set and the test set. Any suitable machine learning classification and/or regression model(s) may be used, with the chosen model(s) depending on the characteristics of the user data and the physiological parameter type(s) to be determined. In an example, a suitable regression model is used for predicting values of each continuous physiological parameter such as heart rate and blood pressure and a suitable classification model is used for predicting values of each non-continues physiological parameter such as movement type (which, for example, takes one of a plurality of predetermined classifications rather than taking a continuous numerical value). As is the norm for machine learning, the skilled person may choose a plurality of different models (and, where applicable, configurations of each model), train each one using the training set and test which of these gives the most accurate classification results using the test set. Example machine learning classification models which could be used include logistic regression, K-Nearest Neighbours (K-NN), Support Vector Machine (SVM), Kernel SVM, Naïve Bayes, Decision Tree Classification, Random Forest Classification, Deep Learning (including Artificial Neural Networks (ANNs) and, in for example the case of image classification, Convolutional Neural Networks (CNNs)) and Reinforcement Learning. Example machine learning regression models which could be used include linear regression (simple or multiple), polynomial regression, Support Vector Regression (SVR), Decision Tree Regression, Random Forest Regression and Reinforcement Learning.

Once trained, the PP model is stored either locally in the memory 202 and/or storage medium 203 of the device 100 or is stored on a remote device (for example a server) accessible to the device 100 via the communication interface 205 (for example using a suitable API). Once trained, the PP model is able to take, as input, an instance of user data it has not seen before (for example a 10 second capture of image, depth map and/or audio data of a viewer captured by the sensor(s) 206) and, as output, determine the likely value of each of the physiological parameter(s) used in the training (for example heart rate, blood pressure and/or movement type).

Once the CT and PP models have been trained, the deployment phase can be implemented. Here, new content 304 (received by content receiver 204) and new sensor input 311 (received by sensor(s) 206)) is collected by the device 100. Content data is extracted from the new content 304 and input to the trained CT model to determine a classification of the new content (step 306). Similarly, user data is extracted from the new sensor input 311 and input to the trained PP model to determine the physiological parameter value(s) of the user data. The extracted content classification and physiological parameter value(s) are then output to a correlation algorithm 307.

This is repeated for a large number of instances of content data and user data to build an initial correlation data set. In an example, the initial correlation data set is determined from, for example, 10,000 instances of user data provided by volunteer viewers of varying characteristics (for example different age groups, gender, health conditions and the like) as they watch varying types of content (for example drama (public service), drama (private service), comedy, sport, etc.). For each instance, the physiological parameter value(s) output by the trained PP model and associated content classification output by the trained CT model are recorded. It is noted the number of instances of user data to generate the initial correlation data set given here represents an example only and that, in reality, the skilled person will choose this value according to that required to statistically represent the population of viewers and to obtain satisfactory performance based on the initial correlation data set.

Once all instances have been recorded, the initial correlation data set assigns expected physiological parameter value(s) for different types of viewer for different type(s) of content. These expected physiological parameter value(s) can then be compared to the actual physiological parameter value(s) of a later viewer for a particular content classification to determine whether the actual physiological parameter value(s) of the viewer are as expected. If they are not, it may be appropriate to alert the user (or a third party). For example, a health warning may be launched (step 314—for example warning the viewer their blood pressure is too high if it exceeds an expected blood pressure for that particular type of viewer for the particular classification of the content they are viewing), a suitable next program may be recommended (step 315—for example a more relaxed program which may help reduce the user's stress), the current content may be changed (step 316—for example if a movie has multiple endings, a calmer, less stressful ending may be selected) or, during the advertisement break, advertisements of potential health benefit to the user may be selected (step 317—for example adverts for lower salt, lower fat food).

Steps 315, 316 and 317 are examples of generating recommended content for the viewer based on their measured and expected physiological parameters. This may be extended to more general content recommendation so that, for example, content likely to be most relevant to the viewer is recommended when the viewer first turns on the TV (for example if the viewer did a very intense exercise video which took their heart rate very high the previous day, a less intense exercise video may be recommended for the current day) and adverts likely to be most relevant to the user are queued to be played in commercial breaks (for example if the viewer has consistently high blood pressure, adverts for lower salt, lower fat food may be chosen over fast food adverts).

The initial correlation data set may also take into account further information such as temporal and/or environmental information 305 (for example time, day, weather) and user set-up information 312 (for example information on a viewer's age, gender and any health condition(s) they have which is input when the user first sets up the device 100) which may affect the expected physiological parameter value(s) for a given viewer. The use of additional information allows the expected physiological parameter value(s) for a given viewer to be further tailored to the specific situation the user is in, thereby improving the performance of the device 100 in determining when to take action and when not to.

For example, it is common for heart rate and blood pressure of a viewer to be different at different times of day. For instance, if a viewer's heart rate and blood pressure are generally lower at night than during the day, the threshold at which an alert is issued if, say, the viewer's heart rate is higher than expected is reduced. This takes into account that a higher heart rate during the day might be seen as normal (when the viewer is typically more active and alert) but might be indicative of a problem if it occurs during the night (when the viewer is typically more relaxed and less alert).

In another example, if a viewer reports they suffer from high blood pressure, the threshold at which an alert is issued if, say, the viewer's blood pressure is higher than expected is increased. This takes into account that spikes in blood pressure might be seen as normal for a viewer who is already aware they have high blood pressure (and who, for example, is already undergoing treatment for the condition) but might be indicative of a problem for a user who is not aware they have high blood pressure (and who therefore is likely to benefit from an alert).

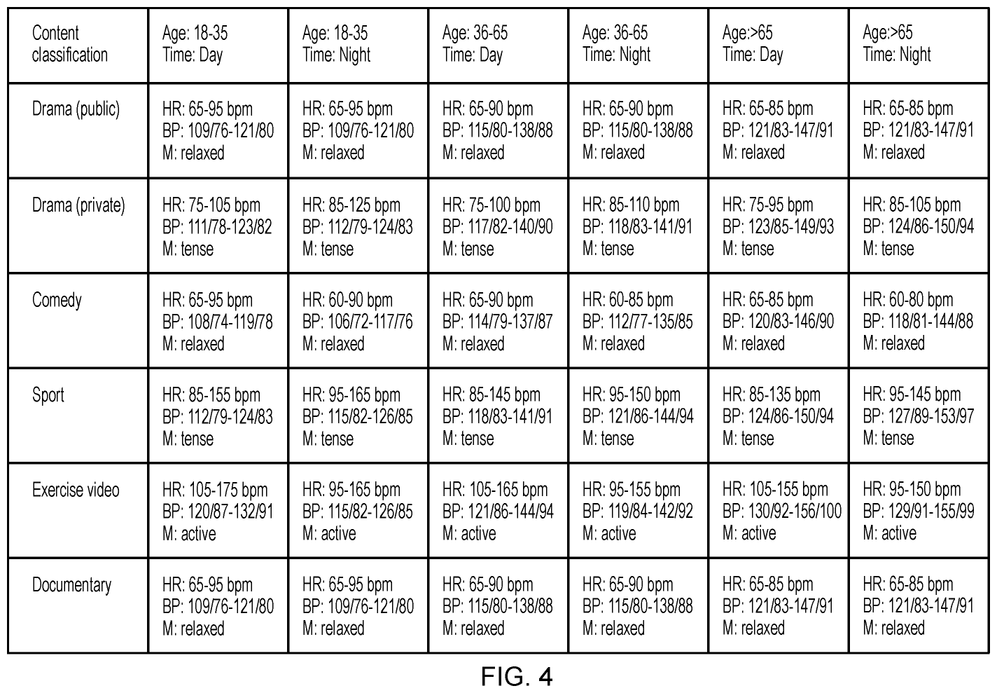

FIG. 4 shows an example initial correlation data set. The initial correlation data set is stored (for example as a look up table) either locally in the memory 202 and/or storage medium 203 of the device 100 or is stored on a remote device (for example a server) accessible to the device 100 via the communication interface 205 (for example using a suitable API).

The left-most column shows the predetermined content classifications. Each new instance of content received by the content receiver 204 is given one of the predetermined content classifications by the trained CT model.

The remaining columns show the expected values of the physiological parameters heart rate (HR), blood pressure (BP) and movement (M). HR and BP are continuous value ranges. M takes one of three discrete classifications. These are “relaxed” (when the user appears inactive (for example sitting) has a relaxed demeanour), “tense” (when the user appears inactive (for example sitting) but has a tense demeanour) and “active” (when the user is physically active (for example following an exercise video)). The expected physiological parameter values are defined for each content classification for each of a number of different viewer classifications. In this example, three different age groups of viewer are given (18-35 years, 36-65 years and greater than 65 years). Furthermore, for each age group, expected physiological parameter values are given for both day (for example 6 am to 6 pm) and night (for example 6 pm to 6 am). This gives a total of six viewer classifications.

Thus, for example, for a viewer who is 19 years old watching comedy at night, their expected heart rate is 60-90 beats per minute (bpm), their expected blood pressure is 106/72-117/76 (systolic pressure/diastolic pressure in mmHg) and their expected movement type is “relaxed”. On the other hand, for a viewer who is 80 years old watching sport during the day, their expected heart rate is 85-135 bpm, their expected blood pressure is 124/86-150/94 mmHg and their expected movement type is “tense”.

The initial correlation data set of FIG. 4 is a simple example for ease of explanation. In reality, there may be a much large number of predetermined content classifications and/or viewer classifications. For example, viewers may be separated into more granulated age groups (for example 18-25, 26-35, 36-44, etc.) and/or more granulated time groups (for example 6:01 am-10:00 am, 10:01 am-2.00 pm, etc.). Additional viewer characteristics may also be used to separate viewers into groups. For example, viewers with medical conditions which are likely to affect one or more the measured physiological parameters (for example high blood pressure) may be classified as belonging to a particular group with a higher average expected blood pressure).

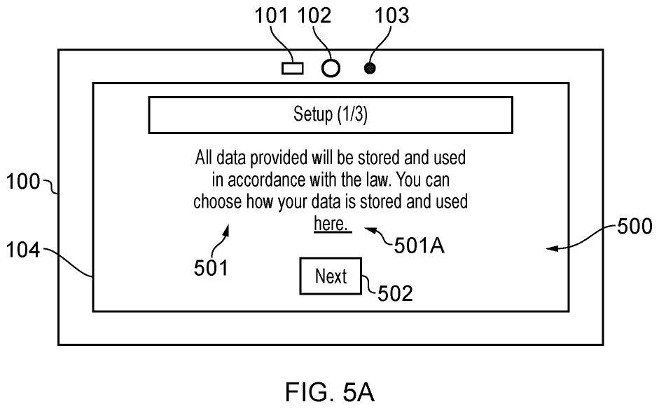

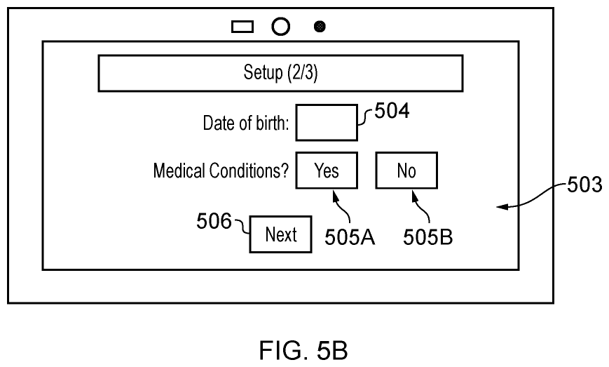

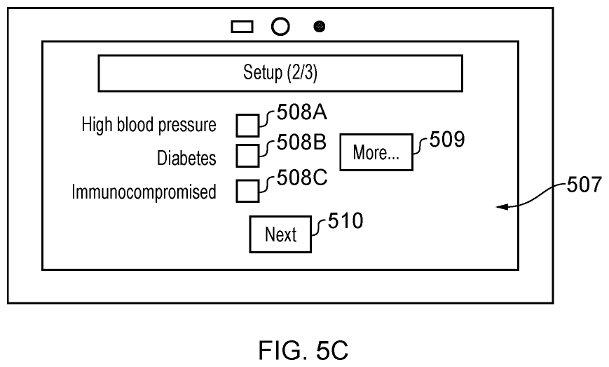

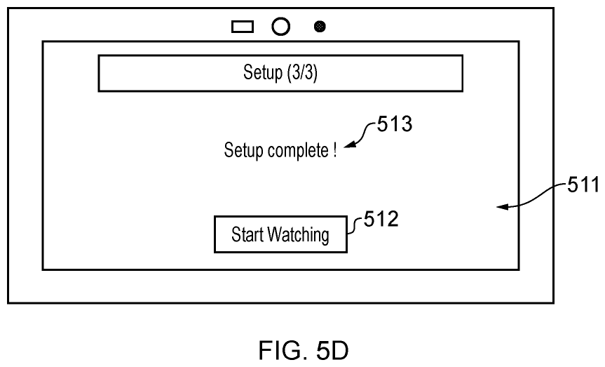

FIGS. 5A-5D show an example set up procedure of the device 100. The set up procedure is carried out when a viewer first purchases and turns on the device, for example. In this example, the device 100 is the TV exemplified in FIG. 1. The viewer is able to control the set up procedure using, for example, a remote control (not shown) of the TV comprising for example cursor buttons, a confirmation button and an alphanumeric keypad. Alternatively, the user may control the set up procedure using voice commands (picked up by the microphone 103 and processed by the processor 202 using a suitable known speech recognition and natural language processing technique, for example).

FIG. 5A shows a first step up screen 500 comprising a textual message 501 informing the viewer that their data (which, as discussed, may contain sensitive health information) will be stored and used lawfully. The user is provided with a link 501A which they may select to view more information about how their data will be used and stored. The user's legal jurisdiction may be detected (for example based on the user's IP address) or entered manually by the user so the user is shown information based on relevant data protection legislation.

To improve user privacy, in an example, all data gathered from the user may be securely stored in encrypted form in a portion of the storage medium 203 not accessible to third parties. In the case that the initial correlation data set is stored on a remote device (for example a server, not shown), the device 100 may only transmit a request indicating the program classification (for example drama (public service), drama (private service), comedy, etc.) and viewer classification (for example 18-35, Day) in order to obtain the relevant expected physiological parameter values to compare with those gathered for the viewer. This means the actual measured parameter values of the viewer do not need to be transmitted by the device 100 to any third party, thus improving user security.

Once the viewer has reviewed and chooses to accept the way in which their data is to be stored and used (i.e. to securely provide the functionality of the present disclosure), they select the “Next” virtual button 502 to proceed to the virtual screen 503 shown in FIG. 2B.

The virtual screen 503 prompts the user to indicate some information about themselves. The information enables the correct viewer classification in the initial correlation data set to be selected for the user. In this example, the viewer is provided with a textbox 504 to enter their date of birth. This enables the age of the viewer to be determined (and hence the correct viewer classification in initial correlation data set). In this example, the viewer is also provided with the opportunity to inform the system about any medical conditions they have. As explained, real life initial correlation data sets may be much more comprehensive than the simplified example given in FIG. 4 and may reflect different expected physiological parameter value(s) for a given viewer depending on whether or not they have a health condition.

Here, if the user does not have a medical condition that they wish to be taken into account, they select the “No” virtual button 505B. They then select the “Next” virtual button 506 and proceed to the completion virtual screen 511 shown in FIG. 5D.

On the other hand, if the user does have a medical condition that they wish to be taken into account, they select the “Yes” virtual button 505A. They then select the “Next” virtual button 506 and proceed to the virtual screen 507 shown in FIG. 5C. Here the use is able to indicate from a picklist the medical condition(s) they have. The selectable medical conditions are those for which there is a corresponding viewer classification in the initial correlation data set. The selectable medical conditions are listed and provided with respective checkboxes to indicate which medical condition(s) are selected. For example, if the user wishes to inform the system they have high blood pressure, the select the checkbox 508A and, in response to the selection, a “tick” or “check” appears in the checkbox 508A. The user may inform the system they have diabetes or are immunocompromised in a similar way by selecting the checkboxes 508B and 508C, respectively. Further medical conditions may be viewed and selected by the viewer selecting the “More . . . ” virtual button 509. The medical conditions shown on the virtual screen 507 are the most commonly selected medical conditions, for example, whereas selecting the “More . . . ” virtual button 509 brings up an exhaustive list of all medical conditions for which there is a corresponding viewer classification in the initial correlation data set. Once the user has selected all relevant medical condition(s), they select the “Next” virtual button 510 and proceed to the completion virtual screen 511 shown in FIG. 5D.

If the user selects multiple medical conditions (for example diabetes AND high blood pressure), there may be a specific viewer classification which takes into account all of those medical conditions. Alternatively, if no specific viewer classification exists for that combination of medical conditions, a most-cautious combination of the physiological parameter values of the viewer classification for each medical condition may be used. For example, if, for the viewer's age group, the expected blood pressure is 120/83-146/90 mmHg if they have high blood pressure and 119/82-142/86 mmHg if they have diabetes, the expected blood pressure may take the highest lower limit (120/83 mmHg for the “high blood pressure” viewer classification) and the lowest higher limit (142/86 mmHg for the “diabetes” classification) to reach an expected blood pressure value range of 120/83-142/86 mmHg for the viewer.

The completion virtual screen 511 includes a textual message 513 informing the viewer the set up procedure is complete. The viewer may then start viewing content by selecting the “Start Watching” virtual button 512. At this point, content the viewer is viewing is received by the content receiver 204 and classified in real time using the trained CT model. Simultaneously, real time user data is collected via the sensor(s) 206 to determine the current physiological parameter values (for example heart rate, blood pressure and movement type) of the viewer using the trained PP model. The current physiological parameter values are then compared in real time to their expected values (as indicated by the initial correlation data set). The health of the viewer can therefore be accurately monitored in real time as they view the content.

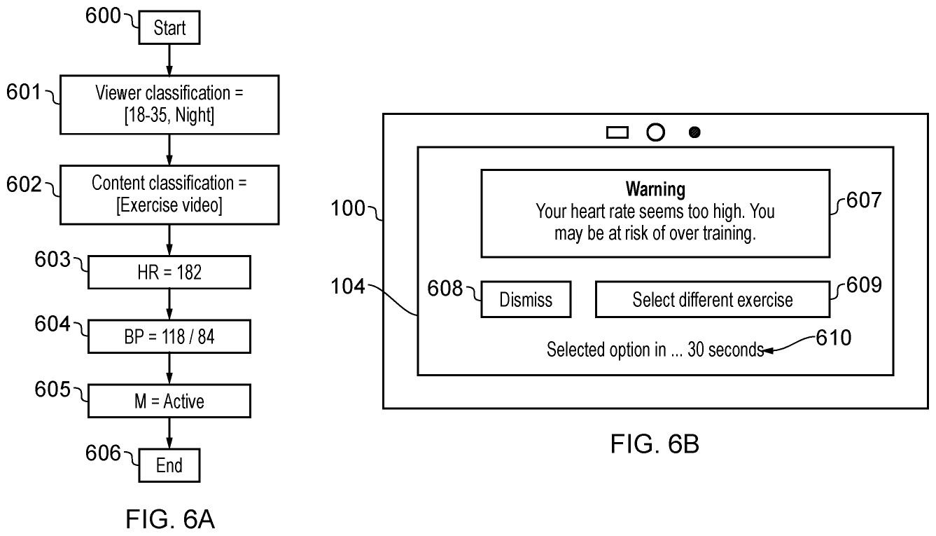

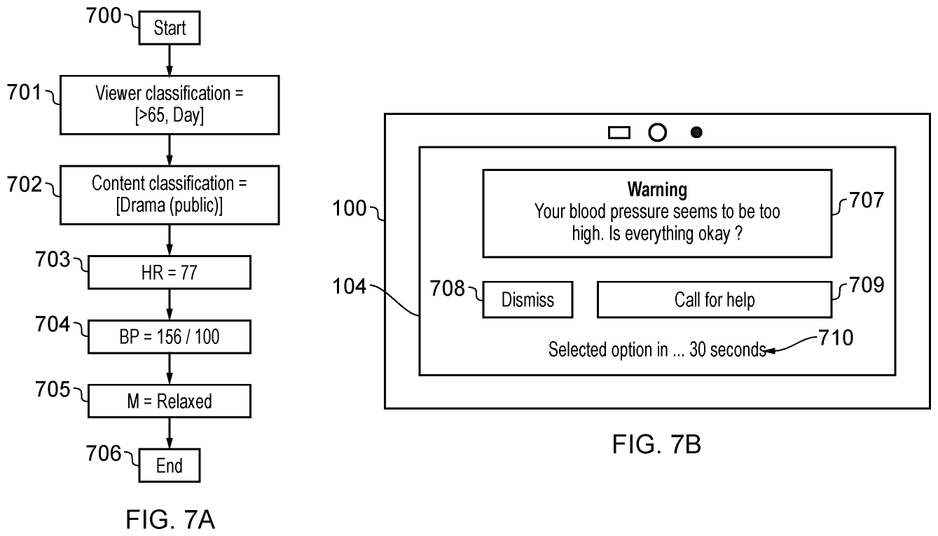

FIGS. 6A-6B and 7A-7B show example implementations of the present disclosure (based on the initial correlation data set shown in FIG. 4).

FIGS. 6A-6B show how the health of an 18-35-year-old viewer watching an exercise video at night may be monitored using the present technique.

The process starts at step 600.

At step 601, the viewer is classified as belonging to viewer classification [18-35, Night].

At step 602, the content is classified as belonging to content classification [Exercise video].

At step 603, the viewer's current heart rate is detected as 182 bpm. Looking at the expected heart rate for viewer classification [18-35, Night] and content classification [Exercise video] in FIG. 4, this is 95-165 bpm. The viewer's heart rate thus lies outside this range and is too high. An alert 607 is thus shown to the viewer indicating their heart rate is too high and that they therefore may be overtraining. The user is given the option to select a different exercise video by selecting the “Select different exercise” virtual button 609. Alternatively, the user may dismiss the alert by selecting the “Dismiss” virtual button 608. If the viewer does not make any selection, the “Select different exercise” virtual button 609 is selected by default and a different exercise video will therefore be selected and displayed to the viewer without them needing to make an active selection. A countdown message 610 (counting down from, for example, 30 seconds) is displayed to inform the viewer about the default selection. This helps nudge the viewer to act in a healthier way whilst ensuring they maintain agency over the decisions they make.

At step 604, the viewer's blood pressure is detected as 118/84 mmHg. Looking at the expected blood pressure for viewer classification [18-35, Night] and content classification [Exercise video] in FIG. 4, this is 115/82-126/85 mmHg. This is therefore within normal range and the device 100 takes no action regarding this parameter.

At step 605, the viewer's movement type is detected as “Active”. Looking at the expected movement type for viewer classification [18-35, Night] and content classification [Exercise video] in FIG. 4, this is also “Active”. This is therefore as expected and the device 100 takes no action regarding this parameter.

The process ends at step 606.

FIGS. 7A-7B show how the health of a greater-than-65-year-old viewer watching a public service broadcast drama at night may be monitored using the present technique. In some jurisdictions (such as the UK and EU), public service broadcast content is particularly trustworthy and therefore the measured physiological parameter values of a viewer watching such content (particularly, for example, an elderly viewer) may be seen as more reliable than with other types of content. This may manifest itself, for example, in narrower expected ranges of physiological parameter values (since the trustworthy nature of the content, lack of loud commercial breaks, etc. means large swings in, say, heart rate or blood pressure are less likely unless there is a medical reason which requires intervention). A higher weighting may also be given to public service broadcast content if, for example, a weighted average of physiological parameter values of the viewer measured over time as the viewer watches different types of content is calculated (rather than only the current value of the physiological parameter values).

It is noted that content including a transition to a loud commercial break may be associated with increased heart rate and/or blood pressure due to such a situation being a shock to the viewer (for example, if the viewer is watching a quiet drama and there is suddenly a loud commercial break when they are not expecting it). For some viewer classifications (for example, younger viewers with no underlying cardiovascular health condition), the increased heart rate and/or blood pressure associated with the shock is unlikely to be significant regarding the health of the viewer. However, for other viewer categories (for example, older viewers and/or those with an underlying cardiovascular health condition), the increased heart rate and/or blood pressure associated with the shock may be significant regarding the health of the viewer. An alert may therefore be appropriate in this latter case.

This may be implemented by, for example, having a content classification indicating content comprising a transition to loud commercial breaks (or likely to do so, for example, private service as opposed to public service broadcast content) and expected physiological parameter value(s) for the different viewer classifications which reflect this. For example, for younger viewers with no underlying cardiovascular health condition, a higher upper limit in the expected heart rate and/or blood pressure range may be appropriate for such content (since the increase in heart rate and/or blood pressure is less likely to be problematic to health and, if the higher upper limit is exceeded, this could be indicative of an underlying cardiovascular health condition which has not yet been detected). On the other hand, for older viewers and/or those with an underlying cardiovascular health condition, a lower upper limit in the expected heart rate and/or blood pressure range may be appropriate for such content (since the increase in heart rate and/or blood pressure is more likely to be problematic to health). This means alerts are more likely to be generated for loud commercial breaks when needed for more vulnerable viewers. At the same time, unnecessary alerts for less vulnerable viewers are alleviated.

The process starts at step 700.

At step 701, the viewer is classified as belonging to viewer classification [>65, Day].

At step 702, the content is classified as belonging to content classification [Drama (public)]. Note this classification denotes “drama” content from a public service broadcaster, thus distinguishing it from the classification [Drama (private)] which denotes “drama” content from a private service broadcaster.

At step 703, the viewer's current heart rate is detected as 77 bpm. Looking at the expected heart rate for viewer classification [>65, Day] and content classification [Drama (public)] in FIG. 4, this is 65-85 bpm. This is therefore within normal range and the device 100 takes no action regarding this parameter.

At step 704, the viewer's blood pressure is detected as 156/100 mmHg. Looking at the expected blood pressure for viewer classification [>65, Day] and content classification [Drama (public)] in FIG. 4, this is 121/83-147/91 mmHg. The viewer's blood pressure thus lies outside this range and is too high. An alert 707 is thus shown to the viewer indicating their blood pressure is too high and asking them if they are okay. The user is given the option to call for help (for example by the device 100 initiating an alert to a third party such as a healthcare professional or predefined contact of the viewer via the communication interface 205) by selecting the “Call for help” virtual button 709. Alternatively, the user may dismiss the alert by selecting the “Dismiss” virtual button 708. If the viewer does not make any selection, the “Call for help” virtual button 709 is selected by default and a call for help is made without the viewer needing to make an active selection. A countdown message 710 (counting down from, for example, 30 seconds) is displayed to inform the viewer about the default selection. This allows the viewer to get help if, for example, they have become unwell and are unable to manually select the “Call for help” virtual button 710 themselves.

At step 705, the viewer's movement type is detected as “Relaxed”. Looking at the expected movement type for viewer classification [>65, Day] and content classification [Drama (public)] in FIG. 4, this is also “Relaxed”. This is therefore as expected and the device 100 takes no action regarding this parameter.

The process ends at step 706.

For viewer classification (for example step 601 of FIG. 6A and step 701 of FIG. 7A), if the device 100 is used by a single viewer (for example a viewer who lives alone), the viewer classification may be stored (for example in the storage medium 203) and automatically selected whenever the viewer turns on the device 100. Alternatively, if the device 100 is used by multiple viewers (for example a family), the viewer classification of each viewer may be stored (for example in the storage medium 203) as part of a viewer profile and, when the device is turned on 100, the viewer may be asked to select a viewer profile to implement. Alternatively, the viewer (and corresponding viewer profile) may be automatically detected (for example by implementing a suitable known facial recognition technique on an image captured by the camera 102). In the latter example, if multiple viewers are detected, a viewer in a most prominent position (for example, the viewer whose face is positioned most centrally in the camera's field of view) may be selected or, for example, if one viewer is an adult and the remaining viewers are children, the adult may be selected.

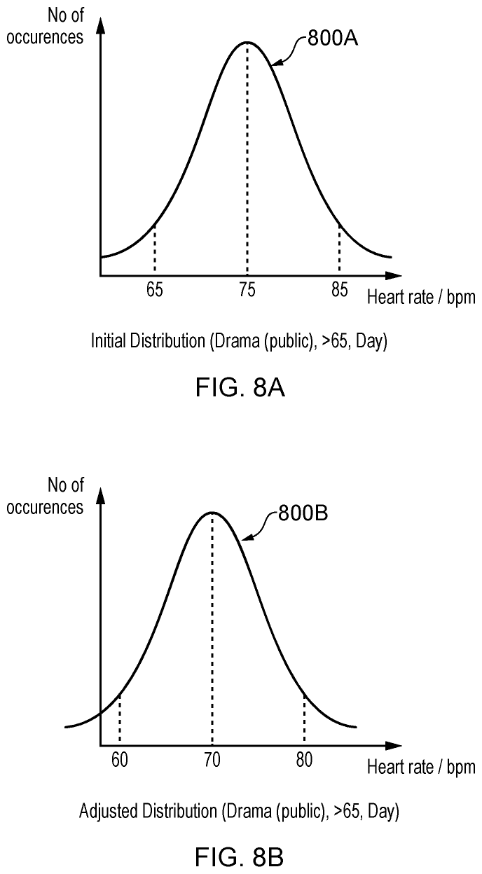

Although the expected physiological parameter value(s) of a given viewer are initially determined based on the initial correlation data set, in an example, the initial correlation data set can be updated for that viewer over time as they use the device 100. This allows the expected physiological parameter value(s) to be tailored for each individual viewer and for the health of that viewer to thus be monitored more accurately. This is exemplified in FIGS. 8A-8B for the expected heart rate value for the content classification [Drama (public)] and viewer classification [>65, Day]. The same principle is applicable to other physiological parameters and different viewer and content classifications. To improve user privacy, the updated correlation data set is stored, for example, in encrypted form in a portion of the storage medium 203 not accessible to third parties.

FIG. 8A shows the determination of the expected heart rate value based on the initial correlation data set. In this example, the expected heart rate value takes values in the range 65-85 bpm based on the distribution of the heart rate values of a sample of viewers of viewer classification [>65, Day] as they watch content with content classification [Drama (public)]. In this case, the mean heart rate value is 75 bpm and the upper and lower limits of 85 bpm and 65 bpm, respectively, are each two standard deviations away from the mean. This is an example and the method of determining the upper and lower limits of an expected parameter value may vary depending on the parameter, for example. Furthermore, in the case of a parameter (for example movement type), the expected parameter value may be the modal value of the viewer sample (rather than a range).

Given this is based on an average and standard deviation of a sample of viewers, however, it may not necessarily best reflect the expected heart rate value of a particular viewer themselves. Thus, as the viewer watches [Drama (public)] content and is monitored by the device 100, the device 100 periodically records the measured heart rate of the viewer. Once a sufficiently large sample has been taken over a sufficient period of time (for example 500 samples over 3 months), the distribution of the recorded heart rate values is determined. This is illustrated in FIG. 8B. Here, it can be seen that the average heart rate of this particular viewer is actually 70 bpm (rather than 75 bpm based on the initial correlation data set) with two standard deviation upper and lower limits of 80 bpm and 60 bpm, respectively (rather than 85 bpm and 65 bpm based on the initial correlation data set). Thus, an updated expected heart rate value of 60-80 bpm for this particular viewer for the content [Drama (public)] is determined. This replaces the previous expected heart rate value for viewer classification [>65, Day] and content classification [Drama (public)] in the updated correlation data set for this particular viewer. This allows the viewer's health to be monitored more accurately, since the expected heart rate value is tailored to the individual viewer rather than based on a sample of the general population of viewers. If the viewer consents, the periodic physiological parameter measurements (for example heart rate) may also be reported as a service to a third party (for example the viewer's doctor) for general health monitoring of the viewer.

In an example, if, for example, a viewer has experienced a serious medical event such as a stroke or heart attack which is unlikely to be reversible (at least in the short term), the user can inform the system. This may (i) update the viewer classification (e.g. if there is a classification for viewers who have experienced the serious medical event in question) and/or (ii) begin a new adjustment process for the user to generate a new updated correlation data set. Thus, for example, if a viewer suffers a heart attack, they are able to inform the system. In response, the viewer classification is changed (e.g. from a classification for viewers with “no medical condition” to a new classification for viewers with a “cardiovascular health condition” and the expected physiological parameter value(s) of the initial correlation data set for that new viewer classification are used. Furthermore, a new adjustment process like that exemplified in FIGS. 8A-8B is started so that, once a sufficient number of new physiological parameter value samples of the viewer have been taken, the expected physiological parameter value(s) of the user are adjusted. This takes into account that the viewer-specific expected physiological parameter value(s) prior to the serious medical event may no longer be appropriate after the serious medical event. This helps ensure accurate health monitoring of the viewer even if they have changing medical circumstances.

Although the examples relate to video content, the present disclosure is not limited to this. For example, the same principles may be applied to audio content only (for example radio broadcasts, audio services provided in TV signals, podcasts, music, internet-delivered streams, pre-curated playlists, user generated playlists and the like) or video content only. More generally, the present disclosure is thus applicable to audio and/or visual content and the viewer may be referred to, more generally, as a content consumer.

In an example, instead of or in addition to being associated with absolute physiological parameter values, it may be associated with relative physiological parameter values. Multiple, directly measurable physiological parameter values (for example, heart rate and blood pressure) may also be combined to provide higher level physiological parameter values (for example, stress level, where a higher stress level is associated with higher heart rate and/or blood pressure and a lower stress level is associated with a lower heart rate and/or blood pressure). For example, an instance of content comprising an “intense suspense scene” might be categorised as “expected stress level +2” and an instance of content comprising a “quiet landscape with animals at rest”, might be categorised as “expected stress level −1”. The expected stress levels are measured relative to a base (zero) level in which no content is shown, for example. In this example, the higher level physiological parameters (for example, stress, positive or negative emotion, etc.) are associated with different content classifications and included in the initial (and any updated) correlation data set, for example.

In an example, content and viewer information like that described can be used to generate more powerful recommendations for content. These recommendations can be user-centred and/or content-centred and may use, for example, a suitable collaborative filtering (CF) technique. In an example implementation, a first matrix for each viewer is generated with each row showing a different viewer reaction (e.g. based on measured physiological parameter value(s) of the viewer) and each column showing a different stimulus (e.g. action scene, scary scene, emotional scene, etc.). Each viewer can then be grouped with other, similar viewers (and thus content liked by one of these viewers may be recommended to the other viewers who are similar to them). Instead or in addition, a second matrix for each instance of content is generated with each row showing a different viewer reaction (e.g. based on measured physiological parameter value(s) of a sample of viewers) and each column showing a different stimulus (e.g. action scene, scary scene, emotional scene, etc.). This allows content to be grouped with other, similar content (and thus similar content to that liked by a viewer may be recommended to that viewer or, if a viewer wishes to explore new types of content they haven't seen before, different types of content to that liked by the viewer may be recommended). For example, highly emotional content (e.g. tragic love stories) is likely to be grouped together where as highly emotion content and action content (e.g. spy thrillers or horror films) is unlikely to be grouped together.

Once the first and/or second matrices are constructed, CF can be used to suggest to user i content j* which is, for example:
unknown to i
appreciated by “neighbours” of i (users with similar taste)
has “distance” between content j* and content j appreciated by user i less than a determined threshold (for example if the viewer wants to view similar content) or, more than a determined threshold (for example if the viewer wants to disrupt the monotony and viewer different content).

In an example in which the content is audio content, playlists of audio content may be curated based on the measured effect(s) they have on the physiological parameter(s) of listeners. The playlist could also take into account the listener's music preferences (for example based on the previous content they have listened to). For example, a “relaxing” audio playlist (comprising, for example, nature sounds and/or subtle harmonies) may be generated for a particular viewer and the select content of the playlist can take into account both (i) the physiological parameter value(s) (for example heart rate and/or blood pressure) of the listener for different audio tracks and (ii) the listener's audio preferences. This allows a bespoke playlist to be made for a listener which allows them to be relaxed whilst enjoying, for example, the genre(s) (e.g. rap, rock, soul, classic, etc.) of music they like. For instance, it allows a relaxing playlist comprising mostly rap music to be generated for one listener (for example, someone who finds rap music relaxing but classical music frustrating) and another relaxing playlist comprising mostly classical music to be generated for another listener (for example, someone who finds classical music relaxing but rap music uncomfortable).

In an example, a convolutional model y=h*x is used in which y is the observed state of a listener (based on one or more physiological parameter values), h is a transfer function modelling the effect of the playlist and x is a relaxed state of the listener (in other words, a hidden “quiet state” we would like to recover). Then, with different playlists associated with different functions h (h1, h2, etc.), an estimate of x can be obtained from the corresponding different listener states y (y1, y2, etc.) actually observed. This allows a relaxed state x of an individual listener to be determined based on the effect different playlists have on their physiological parameter(s) and, through further learning, audio content which correlates to the listeners “relaxed” physiological parameter value(s) can then be chosen as part of a “relaxing” playlist. The convolutional model y=h*x is just an example and, to estimate h, one can use, for example, a Lloyd-Buzo-Gray method, machine learning (for example, deep learning) and/or other algorithm(s).

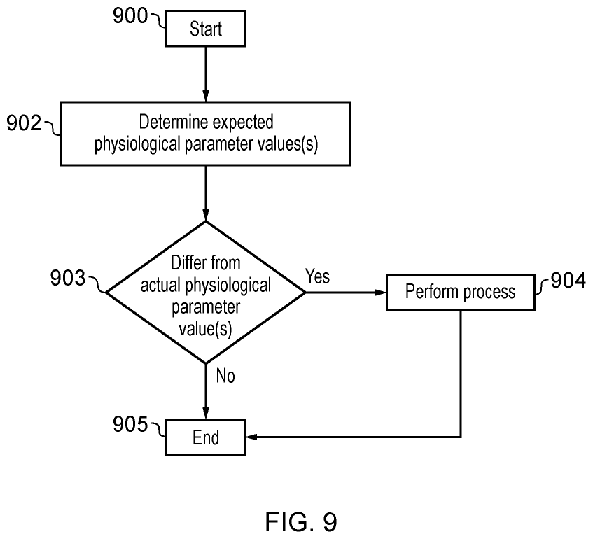

FIG. 9 shows an example method of the present disclosure. The method is implemented under control of the processor 201, for example.

The method starts at step 900.

At step 902, one or more expected physiological parameter values of the consumer when consuming content (for example content received at content receiver 204) are determined based on a classification of the content (for example based on an initial or updated correlation data set), the content being classified based on one or more characteristics of the content.

At step 903, the one or more expected physiological parameter values are compared with one or more corresponding actual physiological parameter values of the consumer when consuming the content (for example as measured by the user data from the sensor(s) 206).

If it is determined that one or more of the one or more actual physiological parameter values differing from its corresponding expected physiological parameter value (for example it lies outside an expected range), the method proceeds to step 904. Otherwise, the method ends at step 905.

At step 904, in response to determining that one or more of the one or more actual physiological parameter values differs from its corresponding expected physiological parameter value, a suitable process (for example displaying a visual alert, providing a content recommendation, alerting a third party and/or one or more other processes addressing the discrepancy between the expected and/or actual physiological parameter values) is performed. The method then ends at step 905.

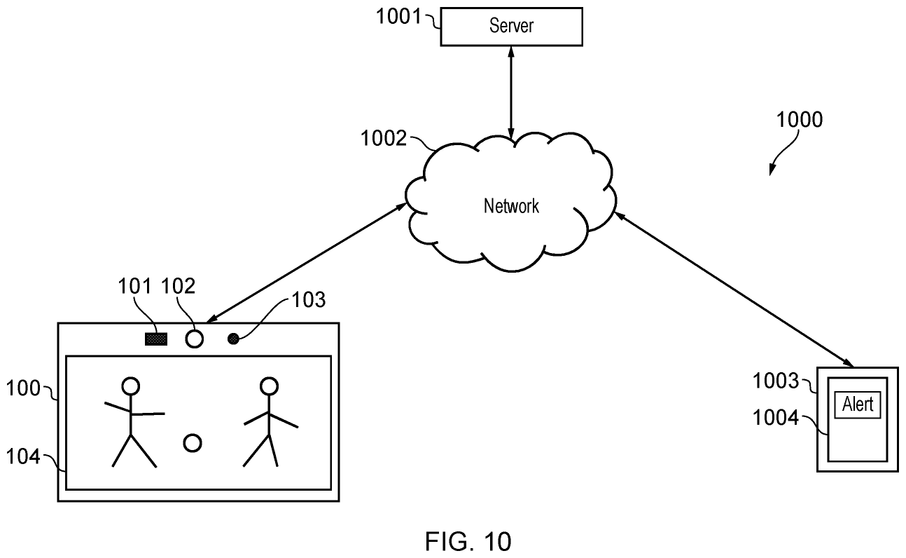

FIG. 10 shows another example of the present disclosure implemented by one or more components of a system 1000. In this example, the system 1000 comprises the device 100. Here, the device 100 is a TV with a screen 104. However, the device 100 may instead be a separate device connectable to a TV, as previously explained. The device 100 comprises the sensors 101, 102 and 103 (which are examples of sensor(s) 206). The device is connected to a network 1002 (for example the internet). The system also comprises a server 1001 connected to the network 1002 and a secondary device 1003 connected to the network 1002. In this example, the secondary device 1003 is a smartphone comprising, among other components, a touch screen 1004. However, the secondary device may be another type of device such as a smartwatch, tablet computer, laptop computer, desktop computer, home personal assistance device (which outputs information via a loudspeaker and/or display and receives input information via voice commands and natural language processing, for example) or the like. In this example, the device 100 is a client of the server 1001.

The principles of the present disclosure are implemented by one or more of the system components.

In an example, the content is classified by the device 100 and/or server 1001. For example, the device 100 classifies received content or sends data indicative of the received content to the server 1001 for the server to undertake the classification. The device 100 and/or server 1001 then implement step 902. For example, the device 100 looks up the initial correlation data set stored on the server 1001 or looks up an updated correlation data set stored on the device 100 itself. The updated correlation data set may also be stored on the server 1001 if, for example, the server is trusted (for instance, only accessible to the user and any trusted third parties such as a medical practitioner).

In an example, the device 100 and/or server 1001 implement step 903. For example, the device 100 itself compares the one or more expected physiological parameter values of the initial or updated correlation data set with the one or more corresponding actual physiological parameter values of the consumer determined from the output of sensors 101, 102 and/or 103). In another example, if the server 1001 is trusted (for instance, only accessible to the user and any trusted third parties such as a medical practitioner), the device 100 may send the sensor data to the server 1001 for the server to undertake the comparison. The sensor(s) whose output is used to determine the actual physiological parameter(s) of the consumer may be comprised as part of the device 100 (for example, the sensors 101, 102 and 103 in FIG. 1) or separate to the device 100 and connected to the device 100 via a suitable interface (for example, a wearable sensor worn by the consumer and connected to the device 100 via a wired or wireless connection, for example a Bluetooth® or Wi-Fi® connection).

In an example, the device 100 and/or server 1001 implement step 904. For example, if the device 100 itself undertakes the comparison and it is determined there is a discrepancy between the expected and actual physiological parameter value(s), the device 100 itself may provide an alert (for example a visual alert on the screen 104) and/or transmit a message over the network 1002 to the secondary device 1004 causing the secondary device to provide an alert (for example a visual alert on the screen 1004). In another example, if the server 1001 undertakes the comparison and it is determined there is a discrepancy between the expected and actual physiological parameter value(s), the server may transmit a message over the network 1002 to the device 100 causing the device 100 to provide an alert (for example a visual alert on the screen 104) and/or transmit a message over the network 1002 to the secondary device 1004 causing the secondary device to provide an alert (for example a visual alert on the screen 1004).

There may be multiple such secondary devices 1003 to which an alert is sent, each of the secondary devices being registered with the consumer in advance. For example, one of the secondary devices may belong to the consumer and the other secondary device(s) may belong to trusted third parties such as a friend, family member or medical professional.

In an example, all steps 902 to 904 are carried out locally on the device 100 itself (with the exception, for example, of obtaining the initial correlation data set from the server 1001). The generated data (for example, the measured actual physiological parameter data and whether or not any alerts have been generated) is then only shared with a third party (for example, by being uploaded to the server 1001 and/or transmitted over the network 1002 to one or more third party secondary devices 1003) with the permission of the consumer. This helps ensure sensitive data about the consumer is secure whilst, with the permission of the consumer, allowing this data to be shared with selected third parties (for example, a friend, family member or medical professional) to help the consumer if necessary.

In example, a record of the actual physiological parameter value(s) determined for the consumer is stored for later retrieval by the consumer and/or a trusted third party (for example, a medical practitioner). The record may be stored on the device 100 itself (for example, in storage medium 203) and/or on the server 1001 (for example, in a storage medium (not shown) of the server 1001). In an example, if the record is stored on the server 1001, suitable credentials (for example, a unique consumer identifier and password) must be provided to the server in order to access the record. The suitable credentials are controlled by the consumer so that only the consumer and, optionally, one or more third parties with whom the consumer decides to share access to the record can access the record. This allows historical physiological data of the consumer to be monitored whilst keeping this data secure. The record may also store related data such as, for example, information indicating the content the consumer was consuming at the time the actual physiological parameter value(s) were recorded and/or the corresponding expected physiological parameter value(s) for the consumer for that content.

Embodiment(s) of the present disclosure are defined by the following numbered clauses:

1. A method of monitoring one or more physiological parameters of a consumer of audio and/or visual content, the method comprising:
determining one or more expected physiological parameter values of the consumer when consuming the content based on a classification of the content;
comparing the one or more expected physiological parameter values with one or more corresponding actual physiological parameter values of the consumer when consuming the content;
performing a process in response to one or more of the one or more actual physiological parameter values differing from its corresponding expected physiological parameter value.

2. A method according to clause 1, wherein the content is broadcast content and is classified based on one or more predetermined broadcast attributes of the content.

3. A method according to clause 2, wherein the one or more predetermined broadcast attributes comprise metadata indicating one or more of a title, a genre, an event table descriptor, an electronic program guide and whether the content is a public service or private service broadcast.

4. A method according to any preceding clause, wherein the one or more physiological parameters comprise one or more of blood pressure, heart rate and movement of the consumer.

5. A method according to any preceding clause, wherein the one or more physiological parameters are determined from one or more of a camera, a microphone, a time-of-flight sensor, an electrocardiograph, ECG, sensor, an oxygen saturation probe, a wearable heart rate monitor, a wearable blood pressure monitor and a wearable respiratory monitor.

6. A method according to any preceding clause, wherein the one or more expected physiological parameter values of the consumer are determined based on physiological information provided by the consumer in advance.

7. A method according to any preceding clause, wherein the one or more expected physiological parameter values of the consumer are determined based on current temporal and/or environmental information.

8. A method according to any preceding clause comprising:
recording a series of actual values of each of one or more physiological parameters of the consumer over time;
adjusting the one or more expected physiological parameter values corresponding to the one or more physiological parameters for which a series of actual values is recorded based on the recorded series of actual values.

9. A method according to any preceding clause, wherein the process comprises outputting an alert to the consumer.

10. A method according to any preceding clause, wherein the process comprises outputting an alert to a third party.

11. A method according to any preceding clause, comprising using the one or more expected physiological parameter values and/or the one or more corresponding actual physiological parameter values to output a content recommendation to the consumer.

12. A method according to clause 11, wherein the content recommendation comprises a content playlist.

13. A device for monitoring one or more physiological parameters of a consumer of audio and/or visual content, the device comprising circuitry configured to:
determine one or more expected physiological parameter values of the consumer when consuming the content based on a classification of the content;
compare the one or more expected physiological parameter values with one or more corresponding actual physiological parameter values of the consumer when consuming the content;
perform a process in response to one or more of the one or more actual physiological parameter values differing from its corresponding expected physiological parameter value.

14. A device according to clause 13 comprising one or more of a camera, a time-of-flight sensor and a microphone, wherein the one or more physiological parameters are determined from one or more of the camera, time-of-flight sensor and microphone.

15. A television comprising a device according to clause 13 or 14.

16. A system for monitoring one or more physiological parameters of a consumer of audio and/or visual content, the system comprising a client and a server comprising circuitry configured to:
determine one or more expected physiological parameter values of the consumer when consuming the content based on a classification of the content;
compare the one or more expected physiological parameter values with one or more corresponding actual physiological parameter values of the consumer when consuming the content;
perform a process in response to one or more of the one or more actual physiological parameter values differing from its corresponding expected physiological parameter value.

17. A system according to clause 16, wherein circuitry of the server is configured to classify the content and determine the one or more expected physiological parameter values of the consumer when consuming the content based on information provided by the client.

18. A system according to clause 16, wherein circuitry of the client is configured to classify the content and determine the one or more expected physiological parameter values of the consumer when consuming the content based on information provided by the server.

19. A system according to any one of clauses 16 to 18, wherein the one or more corresponding actual physiological parameter values of the consumer are determined based on sensor data of one or more sensors in communication with the client.

20. A system according to any one of clauses 16 to 19, wherein circuitry of the server is configured to record information indicative of one or more of the content, the one or more expected physiological parameter values of the consumer and the one or more corresponding actual physiological parameter values of the consumer.

21. A program for controlling a computer to perform a method according to any one of clauses 1 to 12.

22. A storage medium storing a program according to clause 21.

23. A non-transitory storage medium comprising code components which cause a computer to perform a method according to any one of clauses 1 to 12.

Numerous modifications and variations of the present disclosure are possible in light of the above teachings. It is therefore to be understood that, within the scope of the claims, the disclosure may be practiced otherwise than as specifically described herein.

In so far as embodiments of the disclosure have been described as being implemented, at least in part, by one or more software-controlled information processing apparatuses, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more computer processors (for example data processors and/or digital signal processors). The elements and components of any embodiment may be physically, functionally and logically implemented in any suitable way. Indeed, the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to these embodiments. Additionally, although a feature may appear to be described in connection with particular embodiments, one skilled in the art would recognize that various features of the described embodiments may be combined in any manner suitable to implement the present disclosure.

1. A method of monitoring one or more physiological parameters of a consumer of audio and/or visual content, the method comprising:
determining one or more expected physiological parameter values of the consumer when consuming the content based on a classification of the content;
comparing, by circuitry, the one or more expected physiological parameter values with one or more corresponding actual physiological parameter values of the consumer when consuming the content;
performing a process in response to one or more of the one or more actual physiological parameter values differing from its corresponding expected physiological parameter value.

2. The method according to claim 1, wherein the content is broadcast content and is classified based on one or more predetermined broadcast attributes of the content.

3. The method according to claim 2, wherein the one or more predetermined broadcast attributes comprise metadata indicating one or more of a title, a genre, an event table descriptor, an electronic program guide and whether the content is a public service or private service broadcast.

4. The method according to claim 1, wherein the one or more physiological parameters comprise one or more of blood pressure, heart rate and movement of the consumer.

5. The method according to claim 1, wherein the one or more physiological parameters are determined from one or more of a camera, a microphone, a time-of-flight sensor, an electrocardiograph (ECG) sensor, an oxygen saturation probe, a wearable heart rate monitor, a wearable blood pressure monitor and a wearable respiratory monitor.

6. The method according to claim 1, wherein the one or more expected physiological parameter values of the consumer are determined based on physiological information provided by the consumer in advance.

7. The method according to claim 1, wherein the one or more expected physiological parameter values of the consumer are determined based on current temporal and/or environmental information.

8. The method according to claim 1 comprising:
recording a series of actual values of each of one or more physiological parameters of the consumer over time;
adjusting the one or more expected physiological parameter values corresponding to the one or more physiological parameters for which a series of actual values is recorded based on the recorded series of actual values.

9. The method according to claim 1, wherein the process comprises outputting an alert to the consumer.

10. The method according to claim 1, wherein the process comprises outputting an alert to a third party.

11. The method according to claim 1, comprising using the one or more expected physiological parameter values and/or the one or more corresponding actual physiological parameter values to output a content recommendation to the consumer.

12. The method according to claim 11, wherein the content recommendation comprises a content playlist.

13. A device for monitoring one or more physiological parameters of a consumer of audio and/or visual content, the device comprising circuitry configured to:
determine one or more expected physiological parameter values of the consumer when consuming the content based on a classification of the content;
compare the one or more expected physiological parameter values with one or more corresponding actual physiological parameter values of the consumer when consuming the content;
perform a process in response to one or more of the one or more actual physiological parameter values differing from its corresponding expected physiological parameter value.

14. The device according to claim 13 comprising one or more of a camera, a time-of-flight sensor and a microphone, wherein the one or more physiological parameters are determined from one or more of the camera, time-of-flight sensor and microphone.

15. A television comprising a device according to claim 13.

16. A system for monitoring one or more physiological parameters of a consumer of audio and/or visual content, the system comprising a client and a server comprising circuitry configured to:
determine one or more expected physiological parameter values of the consumer when consuming the content based on a classification of the content;
compare the one or more expected physiological parameter values with one or more corresponding actual physiological parameter values of the consumer when consuming the content;
perform a process in response to one or more of the one or more actual physiological parameter values differing from its corresponding expected physiological parameter value.

17. The system according to claim 16, wherein circuitry of the server is configured to classify the content and determine the one or more expected physiological parameter values of the consumer when consuming the content based on information provided by the client.

18. The system according to claim 16, wherein circuitry of the client is configured to classify the content and determine the one or more expected physiological parameter values of the consumer when consuming the content based on information provided by the server.

19. The system according to claim 16, wherein the one or more corresponding actual physiological parameter values of the consumer are determined based on sensor data of one or more sensors in communication with the client.

20. (canceled)

21. A non-transitory storage medium comprising code components, which when executed on a computer, cause the computer to perform the method according to claim 1.

22. (canceled)
